(19) United States
(12) Patent Application Publication
(10) Pub. No.: US 2019/0378619 A1
(43) Pub. Date: Dec. 12, 2019

(54) USING MACHINE LEARNING TO PREDICT HEALTH CONDITIONS

(71) Applicants: Alexander Meyer, Berlin (DE); Dina Zverinski, Berlin (DE)

(72) Inventors: Alexander Meyer, Berlin (DE); Dina Zverinski, Berlin (DE)

(21) Appl. No.: 16/426,969

(22) Filed: May 30, 2019

Related U.S. Application Data
(60) Provisional application No. 62/677,890, filed on May 30, 2018.

(51) Int. Cl. G16H 50/20; G16H 50/70; G06N 3/08; G06N 20/00

(52) U.S. Cl. CPC G16H 50/20; G16H 50/70; G16H 50/30; G06N 20/00; G06N 3/08

(57) ABSTRACT

Technology for predicting health conditions of patients is disclosed. In an example, a first data set comprising features of health data is obtained. A first epoch of training is performed using the first data set. A second data set is generated by applying a bias value to values of a first feature of the first data set. A second epoch of training is performed using the second data set to train the machine learning model. A first set of data comprising static data and a second set of data comprising dynamic data is received, from which a time series data set is derived. A value is determined as absent in the time series data set. The value is assigned using a given data. The time series data set is provided as input to the trained machine learning model to predict health conditions of a patient.

### TECHNICAL FIELD

Aspects and implementations of the present disclosure relate to medical data processing, and more specifically, to predicting health conditions of patients using artificial intelligence methods with a focus on machine learning models.

### BACKGROUND

Machine learning enables computer systems to learn to perform tasks from observational data. Machine learning algorithms may enable the computer systems to learn without being explicitly programmed. Machine learning approaches may include, but not limited to, neural networks, decision tree learning, deep learning, etc. A machine learning model, such as a neural network, may be used in solutions related to health data processing and analysis.

### SUMMARY

The following presents a simplified summary of various aspects of this disclosure in order to provide a basic understanding of such aspects. This summary is not an extensive overview of all contemplated aspects, and is intended to neither identify key or critical elements nor delineate the scope of such aspects. Its purpose is to present some concepts of this disclosure in a simplified form as a prelude to the more detailed description that is presented later.

In an aspect of the present disclosure, a system and methods are disclosed for training a machine learning model (e.g., a neural network, a gated recurrent unit, a support vector machine [SVM], etc.) and using the trained model to predict health conditions of a patient. In one implementation, a system comprises a memory and a processor coupled to the memory, where the processor is to obtain a first data set comprising one or more features of health data associated with one or more patients to train a machine learning model to predict health conditions of patients, perform a first epoch of training using the first data set to train the machine learning model, upon completing the performance of the first epoch, generate a second data set by applying a bias value to values of a first feature of the first data set, and perform a second epoch of training using the second data set to train the machine learning model. In some implementations, the processor may further generate a third data set by removing one or more data points from the first data set, and perform a third epoch of training using the third data set to train the machine learning model. In some implementations, the processor may further generate a fourth data set by modifying a length of a time interval comprising the plurality of time values, and perform a fourth epoch of training using the fourth data set to train the machine learning model.

In one implementation, a method may comprise receiving a first set of data comprising static data for one or more first set of features of health data associated with a patient, receiving a second set of data comprising dynamic data for one or more second set of features of health data associated with the patient, wherein each value corresponding to each feature of the second set of features corresponds to one of a plurality of time values, deriving a time series data set based on the first set of data and the second set of data, determining that a value corresponding to a feature is absent in the time series data set, assigning the value for the feature using a given data, and providing the time series data set as input to a trained machine learning model to predict health conditions of the patient.

In one implementation, a method of treatment may comprise receiving a first set of data comprising static data for one or more first set of features of health data associated with a patient, receiving a second set of data comprising dynamic data for one or more second set of features of health data associated with the patient, wherein each value corresponding to each feature of the second set of features corresponds to one of a plurality of time values, deriving a time series data set based on the first set of data and the second set of data, determining that a value corresponding to a feature is absent in the time series data set, assigning the value for the feature using a given data, providing the time series data set as input to a trained machine learning model to predict health conditions of the patient, predicting that a likelihood of a condition to occur at a future time value is above a predefined threshold, and performing a corrective action comprising one or more of: i) administering an active agent to treat the condition; ii) performing an operative procedure; iii) avoidance of selected active agents; iv) modifying a dose of existing active agent therapy; v) initiation of a diagnostic test; vi) modifying level of patient monitoring; or vii) an additional medical intervention.

Further, computing devices for performing the operations of the above described methods and the various implementations described herein are disclosed. Computer-readable media that store instructions for performing operations associated with the above described methods and the various implementations described herein are also disclosed.

### BRIEF DESCRIPTION OF THE DRAWINGS

Aspects and implementations of the present disclosure will be understood more fully from the detailed description given below and from the accompanying drawings of various aspects and implementations of the disclosure, which, however, should not be taken to limit the disclosure to the specific aspects or implementations, but are for explanation and understanding only.

FIG. 1 depicts an illustrative computer system architecture, in accordance with one or more aspects of the present disclosure.

FIG. 2 depicts a flow diagram of one example of a method for training a machine learning model, in accordance with one or more aspects of the present disclosure.

FIG. 3 depicts a flow diagram of one example of a method for predicting a likelihood of a health condition to occur, in accordance with one or more aspects of the present disclosure.

FIG. 4 depicts an example of generating a time series data set of features, in accordance with one or more aspects of the present disclosure.

FIG. 5 depicts an example of predicting a health condition using machine learning, in accordance with one or more aspects of the present disclosure.

FIG. 6 depicts a block diagram of an illustrative computer system operating in accordance with one or more aspects of the disclosure.

### DETAILED DESCRIPTION

In health care setup, many different types of measurements and data, including but not limited to, physiological, clinical, historic, demographical, procedural and diagnostics related measurements and data, are collected and recorded for patients. The amount of concurrently incoming health data in high volume and challenging environment, such as intensive care units, can overwhelm health care professionals and may lead to treatment delays or clinical errors. Application of machine learning in such environments can provide highly accurate and timely decision making capabilities for supporting health care needs. However, there are various challenges in applying machine learning in terms of medical data processing and analysis.

Health care related data may come in various formats. The data may be collected using various means. For example, medical devices may be used in some instances to collect some medical data for patients in electronic formats. In some setup, health care professionals may collect health related data and manually record the data for tracking purposes. In some instances, these manually recorded data may be entered in an electronic data collection system. In some instances, a combination of automatic electronic data collection and a manual data collection may be used. The different variations and combination of medical data collection may lead to inconsistencies in the data being collected. For example, measurements may be missing for some medical features of a patient while for another patient the measurements may have been collected. Some values may be missing for the same patient at a given time while the values corresponding to a different time may be present. In some cases, the type of value entered for a feature may be different than the expected type of value. Units of measurements in one instance may be different from units of measurements in another instance.

Data recording in electronic health record (EHR) systems is usually designed and optimized for reporting, liabilities, and billing purposes. Much of the data may not be in a suitable form for use by machine learning systems. Data may be organized and stored across a variety of systems, which require integration and harmonization of the data prior to use in an artificial intelligence systems. Missing or inconsistent data attributes may lead to significant levels of error or noise to the analysis and decision making process of an artificial intelligence system.

In addition, a machine learning model may be provided with sample data set as training sets of data which the machine learning model can learn from. The larger and varied the training sample, the better it is possible to train a machine learning model. However, providing a machine learning model with varied and adequate number of sample training data is often a difficult task due to the limited availability of such data in health care systems.

Disclosed herein are aspects and implementations of an automated system that is capable of producing a larger number of training samples by using a number of augmentation (e.g., expansion, increase, etc.) techniques unique and appropriate for medical data (e.g., health data). Techniques may involve using a set of existing data and presentation of the data set from a new perspective without compromising the authenticity of the existing data set. In one implementation, a time series data set may be used where features of health data associated with patients for different time intervals are used to train a machine learning model for use in a prediction of future health conditions of patients. The time series data set may be augmented to generate a new training set of data for further training the model. In one example, the augmentation technique may involve applying a bias (e.g., a difference) value to values of a particular feature of the data set. The bias value may be selected such that the values of the feature remain within a clinically acceptable range of values for the feature after the bias value is applied. In another example, the augmentation technique may involve removing one or more data points from the data set. For example, a particular value of a feature may be removed, a whole feature may be removed, values corresponding to a particular time interval may be removed, etc. In another example, the augmentation technique may involve time warping, or modifying (e.g., increasing, decreasing, etc.) a length of time interval of the time series data set.

After the machine learning model has been trained, a time series data set associated with a patient may be provided as an input to the trained machine learning model to predict a likelihood of a particular condition to occur for a particular patient at a future time. The future time range may vary by prediction target or based on the condition to be predicted. For example, a clinically meaningful time horizon for bleeding prediction may be for within the next 24 hours. In another example, for circulatory arrest the future time range may be 2 hours, while for renal failure, the future time horizon may be three to seven days. In one implementation, the time series data set for the input may be derived using a static data set and a dynamic data set. The static data set may comprise static data (e.g., data not changed over time interval of interest) for a number of features of health data associated with the patient. The dynamic data set may comprise dynamic data (e.g., data that may change over time) for a number of other features of health data associated with the patient. The dynamic data may comprise values of the features corresponding to different time values (e.g., time intervals) in a time series. The static data may be replicated for each time value in the time series such that the static data and the dynamic data may be combined in one time series data set. In addition, it may be determined that a value corresponding to a feature may be absent (e.g., missing, empty, null, etc.) within the time series data set. A given data may be used to assign to the absent value for the feature such that the time series data set input may be complete. The time series data set may then be provided to the trained machine learning model to predict health conditions of the patient for future time intervals.

After the machine learning model is used to predict that a likelihood of a particular condition to occur at a future time is above a predefined threshold of acceptable likelihood, health care professional attention may be triggered and/or a corrective action may be performed. In one embodiment, the corrective action may comprise administering an active agent to treat the condition. In another embodiment, the corrective action may comprise performing an operative procedure on the patient. In another embodiment, the corrective action may comprise avoidance of selected active agents. In another embodiment, the corrective action may comprise modifying (e.g., increasing, decreasing, etc.) of a dose of an existing active agent therapy. In another embodiment, the corrective action may comprise initiation of a diagnostic test to further diagnose or confirm the predicted conditions or additional conditions. In another embodiment, the corrective action may comprise modifying level of patient monitoring. Other types of additional medical interventions may be performed to treat the condition predicted using the machine learning model.

Aspects of the present disclosure thus provide technology by which currently available health data of patients can be used with machine learning systems to predict future medical conditions of patients. The technology allows medical data to be curated and harmonized such that the data is usable within health related machine learning models. The technology provides means for using a set of training data to generate larger data sets for better performance and accuracy of predictions of health conditions. The technology provides for solving particular problems associated with health records, such as missing and/or inconsistent values, to arrive at predictions of health conditions using machine learning. The technology allows for flexibility in terms of treating a patient by the patient's health care personnel. Accordingly, accuracy and efficiency of health condition predictions using machine learning techniques may be improved using the aspects described in the present disclosure.

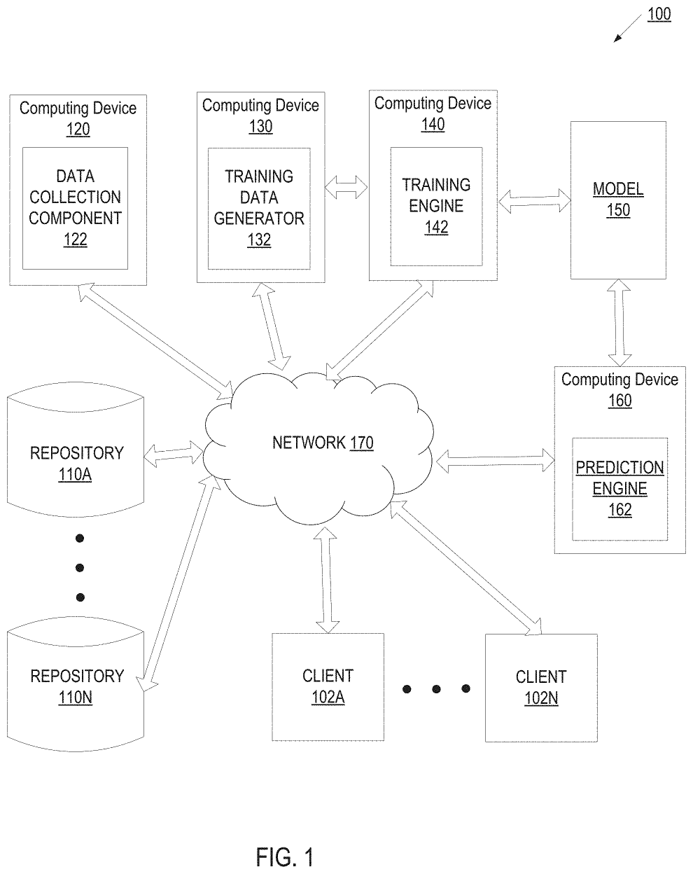

FIG. 1 illustrates an illustrative system architecture 100, in accordance with one implementation of the present disclosure. The system architecture 100 includes one or more computing devices 120, 130, 140, 160, one or more repositories 110A through 110N, and client machines 102A-102N connected to a network 170. In some examples, computing devices 120-160 may be hosted using a cloud computing environment. Network 170 may be a public network (e.g., the Internet), a private network (e.g., a local area network (LAN) or wide area network (WAN)), or a combination thereof. The various computing devices may host components and modules to perform functionalities of the system 100. System 100 may include a data collection component 122, a training data generator 132, a training engine 142, a model 150, and a prediction engine 162.

The client machines 102A-102N may be personal computers (PCs), laptops, mobile phones, tablet computers, set top boxes, televisions, digital assistants or any other computing devices. The client machines 102A-102N may run an operating system (OS) that manages hardware and software of the client machines 102A-102N. In one implementation, the client machines 102A-102N may be used to monitor and predict health conditions of patients.

Computing device 120 may be a rackmount server, a router computer, a personal computer, a portable digital assistant, a mobile phone, a laptop computer, a tablet computer, a camera, a video camera, a netbook, a desktop computer, a media center, or any combination of the above. Computing device 120 may include an application specific integrated circuit (ASIC), a field programmable gate array (FPGA), a digital signal processor (DSP), other types of Integrated Circuits (IC), a distributed computing system, a cluster of machines, blockchain environment, or other compound combination of machines. Computing device 120 may include a data collection component that is capable of collecting health data (e.g., physiological measurements, health attributes, treatment, conditions, procedures, etc.) from various data sources, including repositories 110A-N (e.g., using software agents, etc.). For example, data collection component 122 may connect to hospital databases, other types of Electronic Health Records (EHR) systems, physician data stores, patient portals, etc.

Repositories 110A-N may include persistent storage that is capable of storing a number of data types as well as data structures to tag, organize, and index health related data. Repositories 110A-N may be hosted by one or more storage devices, such as main memory, magnetic or optical storage based disks, tapes or hard drives, NAS, SAN, and so forth. In some implementations, repositories 110A-N may be network-attached file server, while in other implementations, repositories 110A-N may be other types of storage such as an object-oriented database, a graph based database, a document store, a key value store, a relational database, or combination thereof, that may be hosted by the computing device 120 or one or more different computing devices coupled to the computing device 120 via the network 170. Repositories 110A-N may include repositories associated with hospital databases, other types of Electronic Health Records (EHR) systems, physician data stores, patient portals, various text documents such as surgical reports or imaging study reports, raw imaging data, genomic data, etc. The data stored in the repositories may include text data, numeric data, imaging data, structured data, etc.

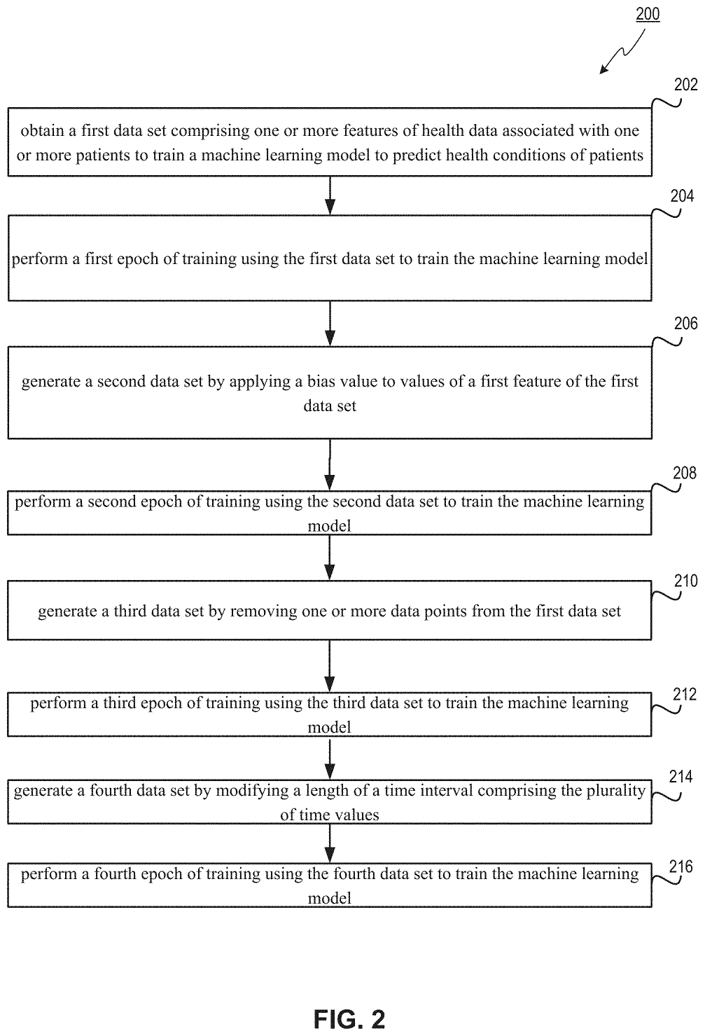

Computing device 130 may include a training data generator 132 that is capable of generating training data (e.g., a set of training inputs and target outputs) to train a machine learning model 150 for use with a prediction engine 162. Some operations of training data generator 132 are described in detail below with respect to FIG. 2.

In addition to generating training data for the prediction engine 162, the training data generator may also use additional machine learning models to identify and add labels for outcomes based on the training data. As a background, many systems may collect measurements (e.g., data) related to patients without labeling the outcome (e.g., medical condition) associated with the measurements. For example, a set of features x, y, and z with certain values or range of values for the respective features may be related to an outcome of “mortality.” In another example, a set of features q, r, s, z with particular values for the respective features may be related to an outcome of “bleeding.” In another example, a set of features a, b, c with particular values for the respective features may be related to an outcome of “cardiac arrest.” Each of these outcomes may be explicitly identified or labeled in the system containing these features associated with the particular features and values. However, in many medical systems, some or all labels may not be identified or available within the systems. For some outcomes, the labels may be easy to predict (e.g., mortality) based on the features and values, while for some outcomes, the labels may not be easy to predict based on the features and values. In order to train a machine learning model, such as, a neural network, to predict future outcomes, the neural network is to be trained with data set that clearly identifies training input (e.g., features and values) and target output (the outcome to be predicted). The training data generator 132 may provide the features and corresponding values as the training input. However, the neural network may not be trained if the target output is not identified in the training data set. To resolve this issue, the training data generator may use additional neural networks (or other machine learning algorithms) to detect the outcomes based on features and values and add labels for outcomes with the training set of data for the model for the prediction engine based on the neural networks.

The training data generator may have a label detector module to detect and generate the labels for the outcomes. The label detector may also be independent of the training data generator and feed the results to the training data generator. The label detector may reside within computing device 130, or a separate computing device. The label detector may use a machine learning algorithm, such as, neural network, Random Forest, SVM, etc., with training set of data to detect outcomes. Additionally, other techniques, such as, natural language processing, may be used to extract labels from unstructured text data (e.g., physican's notes, finding reports, written history, imaging study reports, etc.). The detection of the outcomes at this stage may be distinct from the prediction of likelihood of outcomes based on time series data. The machine learning algorithm in this stage may be provided with the training set of data including a set of features corresponding to a set of values and a known outcome. The machine learning algorithm may learn the patterns from the features, values, and known outcomes and be able to detect similar types of outcomes when provided with comparable set of features and corresponding values. In some scenarios, the initial set of training data may need to be carefully vetted to verify that the input data is as close to being correct as possible. This may include manually assessing plurality of raw medical data (e.g., identifying precise time range of resuscitation to declaring cardiac arrest) that can be initially provided to the machine learning algorithm to train the label detector. From the initial training, the machine learning algorithm may learn to identify the patterns using robust training data set and detect the outcomes accurately.

Once the label detector is sufficiently trained, the label detector may be provided with the features (e.g., “RR_{systolic}”) and values (e.g., “148”) that are made available using the training data generator. The label detector may detect an outcome using the trained machine learning model and produce a label (e.g., “cardiac arrest,” “renal failure,” etc.) that is to be stored along with the training data set for the model 150. For example, the generated labels may be added to an outcome column of a table that comprises the features and the values based on which the labels were detected. In some examples, the outcome column may already exist. The outcome column may contain no values for a particular row, or may contain some value. The generated label may be added as a value for the outcome column if no values already exist. If a value already exists, the generated label may be added to replace the existing value to modify the outcome column value. Once the outcomes are detected and labels are generated and added for the features and corresponding values, the training data set for the model 150 may be complete with both inputs and outputs so that the model 150 can be trained for prediction engine 162.

Computing device 140 may include a training engine 142 that is capable of training a machine learning model 150. The machine learning model 150 may refer to the model artifact that is created by the training engine 142 using the training data generated by training data generator 132. The training engine 142 may find patterns in the training data that map the training input to the target output (the answer to be predicted), and provide the machine learning model 150 that captures these patterns. The machine learning model may be composed of, e.g., a single level of linear or non-linear operations (e.g., a support vector machine [SVM] or may be a deep network, i.e., a machine learning model that is composed of multiple levels of linear or non-linear operations. An example of a deep network is a neural network with one or more hidden layers, and such machine learning model may be trained by, for example, adjusting weights of a neural network in accordance with a backpropagation learning algorithm or the like. For convenience, the remainder of this disclosure will refer to the implementation as a neural network, even though some implementations might employ an SVM or other type of learning machine instead of, or in addition to, a neural network. In one aspect, the training set is obtained from computing device 130.

The training engine 142 may itself use a neural network to train the machine learning model. The training engine 142 may train the machine learning model 150 using a full training set of data multiple times. Each training cycle using the full training set of data may be referred to as an epoch. Each training cycle or an epoch may use one forward pass and one backward pass of all training data in the training set. As a background, neural networks may consist of layers of computational units to hierarchically process data, and feeds forward the results of one layer to another layer, extracting a certain feature from the input. In a neural network, when an input vector is presented to the network, it may be propagated forward (e.g., a forward pass) through the network, layer by layer (e.g., computational units) until it reaches the output layer. The output of the network is then compared to the desired output (e.g., the label), using a loss function. The resulting error value is calculated for each of the neurons in the output layer. The error values are then propagated from the output back through the network (e.g., a backward pass), until each neuron has an associated error value that reflects its contribution to the original output. Multiple epochs of training may be performed using one or more training data sets. Some operations of training engine 142 are described in detail below with respect to FIG. 2.

Computing device 160 may include a prediction engine 162 that is capable of providing health data set associated with a patient as input to trained machine learning model 150 and running trained machine learning model 150 on the input to predict future medical conditions of the patient as output. In one implementation, prediction engine 152 is capable of processing health data to provide a time series data set as input to the model 150. In one implementation, prediction engine 152 is also capable of imputing (e.g., assigning) values to features that are missing values.

It should be noted that in some other implementations, the functions of computing devices 120, 130, 140, and 160 may be provided by a fewer number of machines. For example, in some implementations computing devices 130 and 140 may be integrated into a single computing device, while in some other implementations computing devices 130, 140, and 160 may be integrated into a single computing device. In addition, in some implementations one or more of computing devices 120, 130, 140, and 160 may be integrated into a comprehensive medical platform.

In general, functions described in one implementation as being performed by the comprehensive medical platform, computing device 120, computing device 130, computing device 140, and/or computing device 160 can also be performed on the client machines 102A through 102N in other implementations, if appropriate. In addition, the functionality attributed to a particular component can be performed by different or multiple components operating together. The comprehensive medical platform, computing device 120, computing device 130, computing device 140, and/or computing device 160 can also be accessed as a service provided to other systems or devices through appropriate application programming interfaces.

FIG. 2 depicts a flow diagram of one example of a method 200 for training a machine learning model, in accordance with one or more aspects of the present disclosure. The method is performed by processing logic that may comprise hardware (circuitry, dedicated logic, etc.), software (such as is run on a general purpose computer system or a dedicated machine), or a combination thereof In one implementation, the method is performed by computer system 100 of FIG. 1, while in some other implementations, one or more blocks of FIG. 2 may be performed by one or more other machines not depicted in the figures. In some aspects, one or more blocks of FIG. 2 may be performed by training data generator 132 of computing device 130. In some aspects, one or more blocks of FIG. 2 may be performed by training engine 142 of computing device 140.

For simplicity of explanation, methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

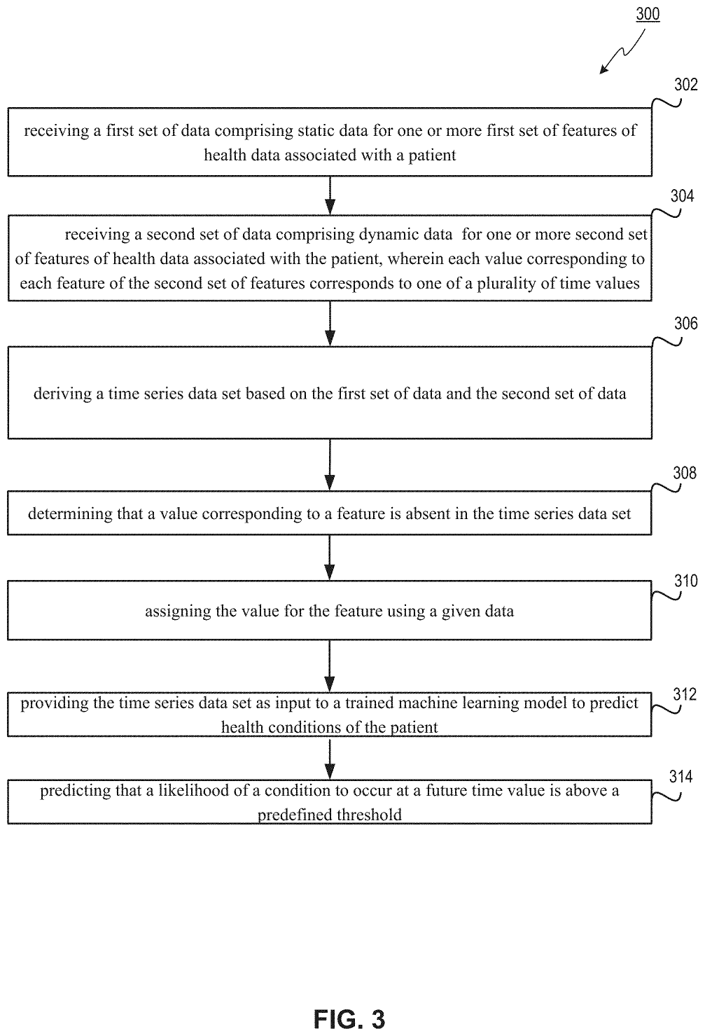

Method 200 begins with obtaining training data for a machine learning model. At block 202, a first data set is obtained, the first data set comprising one or more features of health data associated with one or more patients to train a machine learning model to predict health conditions of patients. In one implementation, the first data set may be obtained in view of data collection component 122 as shown in FIG. 1. The first data set may be derived by filtering data collected by data collection component 122. The data may be filtered so that data relevant for model 150 using training engine 142 is selected within the first data set. In some examples, the obtained first data set may comprise a time series data set. For example, one or more features of health data associated with the one or more patients corresponding to a plurality of time values may be obtained as a time series data set. The time series data set may be derived using a static data set and a dynamic data set. The static data set may comprise static data (e.g., data not changed over time interval of interest) for a number of features of health data associated with the patient. The dynamic data set may comprise dynamic data (e.g., data that may change over time) for a number of other features of health data associated with the patient. The dynamic data may comprise values of the features corresponding to different time values (e.g., time intervals) in a time series. The static data may be replicated for each time value in the time series such that the static data and the dynamic data may be combined in one time series data set for training the model 150. Derivation of time series data is described in further detail with respect to FIG. 3. For example, the handling of missing data values for a feature by imputing the values as described in FIG. 3 also apply to handling of missing data for the time series data described with regards to block 202.

At block 204, a first epoch of training may be performed using the first data set to train the machine learning model. The first epoch may correspond to a first forward pass and a first backward pass, as described above, that are associated with training the machine learning model using each data point of the first data set. During each epoch, a full set of training data may be utilized to train a machine learning model. Thus, during the first epoch, a full set of data for the first data set may be used to train the model 150. Even though block 204 depicts performing a single epoch of training using the first data set, multiple epochs of training may be performed using the first data set to refine and improve the learning prediction capabilities of model 150.

At block 206, a second data set may be generated. In some implementations, training data generator 132 may generate the second data set. Generating the second data set may involve an augmentation technique. The augmentation technique for generating the second data set may involve applying a bias value to values of a first feature (or any feature) of the first data set. A bias value may correspond to a difference applied to the values of the first feature (or any feature) of the first data set. In one example, the bias value may be applied by appending a random value to the values of the first feature (or any other selected feature) of the first data set. In an example, the random value may be constricted within a given range of values. The random value may be a value that is below a first threshold value defined within the system. In some examples, the random value may be within a specified number of standard deviation of the values of the first feature (or any selected feature). In some examples, the bias value may be selected such that applying the bias value results in each of the values of the first feature (or any feature) to remain within a clinically acceptable range of values for the first feature (or any selected feature). In some examples, the bias value may be selected such that it reflects a typical pattern caused by an artifact. In some examples, a set of clinically plausible values may be formulated for the first feature. The values of the first feature of the first data set may be replaced using one or more random values from the set of clinically plausible values. In an example, all arterial blood pressure values decline to about 45 mmHg for one minute from when a blood gas sample is taken. For the time interval corresponding to one minute from when the sample is taken, this value (45 mmHg) may be randomly applied to some of the values for some patients.

At block 208, a second epoch of training maybe performed using the second data set to train the machine learning model. The second epoch may correspond to a forward pass and a backward pass, as described above, that are associated with training the machine learning model using each data point of the second data set. Even though block 208 depicts performing a single epoch of training using the second data set, multiple epochs of training may be performed using the second data set to refine and improve the learning prediction capabilities of model 150.

At block 210, a third data set may be generated. In some implementations, training data generator 132 may generate the third data set. Generating the third data set may involve an augmentation technique. The augmentation technique for generating the third data set may involve by removing one or more data points from the first data set, or any other data set previously generated by the training data generator 132 (e.g., a base data set off of which to generate the third data set). In some example, a particular value of a feature of the first data set (or the base data set) may be removed to generate the third data set. In some example, each value of an entire feature may be removed from the first (e.g., base) data set. This may simulate a setting where an entire feature is not present for a patient or a set of patients. In some examples, values of all features of the first (e.g., base) data set corresponding to a specified time interval may be removed. This may, for example, simulate a situation when data for a patient was not available or collected, such as moving the patient from one bed to another, etc. In some examples, one or more values of a feature of the first (e.g., base) data set corresponding to each of the one or more patients may be removed randomly. In some implementation, a combination of one or more of the above examples may be used to remove data from the base set to generate a new training set.

At block 212, a third epoch of training maybe performed using the third data set to train the machine learning model. The third epoch may correspond to a forward pass and a backward pass, as described above, that are associated with training the machine learning model using each data point of the third data set. Even though block 212 depicts performing a single epoch of training using the third data set, multiple epochs of training may be performed using the third data set to refine and improve the learning prediction capabilities of model 150. Additionally, as described initially, the operations of blocks 210 and 212 may be performed in a different sequence than depicted in FIG. 2. For example the operations of blocks 210 and 212 may be performed prior to performing the operations of blocks 206 and 208.

At block 214, a fourth data set maybe generated. In some implementations, training data generator 132 may generate the fourth data set. Generating the fourth data set may involve an augmentation technique. The augmentation technique for generating the fourth data set may involve modifying a length of a time interval comprising the plurality of time values. In some example, the length of the time interval may be preferred to be increased. In some example, the length of the time interval may be preferred to be decreased. For example, data collected for a feature that were collected from different systems may have been measured or collected at different intervals. A first system may contain data that is measured or available at, for example, a 30 minute interval, while another system may contain data that is measured or available for every one minute interval. Using an augmentation technique to modify time intervals, in one scenario, the frequency of data may be decreased. That is, for both systems, data may be used for each 30 minute interval. In that case, the measurements available at every minute intervals may be skipped to match with the 30 minute intervals of the first system. Alternatively, frequency of data may be increased, where data from the every minute interval is used. For this technique, since the system with the 30 minute interval may not have data available at every minute interval, the latest (e.g., most recent as compared to the current time value) valid measurements for the feature may be used for the measurements that are missing for the time value.

In some examples, linear interpolation may be used to reconstruct a dense time series back again. For example, using linear interpolation, a mathematical function may be generated from the measured feature values against time interval values. Once the mathematical function is generated, the function can be used to generate feature values for time values that do not have measured data, thus increasing the length of the time interval duration. The function may be used to generate feature values for all time values in a given interval for the augmented data. In an example, a system may contain a particular feature values for a patient for every minute from 0 minutes to 120 minutes (e.g., time values). Using the available relationship between, or curve of, the time values against feature values, a correlation may be identified and a function may be generated for the relationship pattern. The function can then be used to generate feature values for a time value that was not contained in the system, such as feature values corresponding to time values of 121, 122, 180, etc. The function can also be used to generate feature values for all time values in the time range of 0 to 180 minutes. In this way, the duration or time range is increased using interpolation and mathematical function generation. Additionally or alternatively, the function may also be used to decrease the time range. For example, once the function is available based on the time values of 0 to 120 minutes, the time range to use in a dataset may be reduced to 0 to 60 minutes and instead of using the measured feature values, the function can be used to generate values for the 0 to 60 minute time range so that the pattern may still be maintained.

At block 216, a fourth epoch of training using the fourth data set to train the machine learning model may be performed. The fourth epoch may correspond to a forward pass and a backward pass, as described above, that are associated with training the machine learning model using each data point of the fourth data set. Even though block 216 depicts performing a single epoch of training using the fourth data set, multiple epochs of training may be performed using the fourth data set to refine and improve the learning prediction capabilities of model 150. Additionally, as described initially, the operations of blocks 214 and 216 may be performed in a different sequence than depicted in FIG. 2. For example blocks 214 and 216 may be performed prior to performing the operations of blocks 206 and 208, or the operations of blocks 210 and 212.

Alternatively, instead of performing each epoch of training with a different set of augmented data, the second, third, and fourth data sets may be generated using augmentation techniques described above, and the first, second, third, and fourth data sets may be combined in one combined data set. The machine learning model may then be trained using the combined data set. In some examples, the combined data set may include one of the first, second, third, or the fourth data set, or a combination thereof. Multiple epochs of training may be performed using the combined data set. The number of epochs to train the model may be determined based on results of running validation sets using the combined data set after each epoch is performed. Thus, the alternative implementation may begin by obtaining a first data set comprising one or more features of health data associated with one or more patients to train a machine learning model to predict health conditions of patients. The one or more features may correspond to a plurality of time values. A second data set may be generated. The second data set may be generated by using one or more augmentation techniques. The augmentation techniques may include applying a bias value to values of a first feature of the first data set, removing one or more data points from the first data set, and/or modifying a length of a time interval comprising the plurality of time values. The second data set may include data sets generated using the augmentation techniques and combining the data sets. Next, training of the machine learning model may be performed using a combined training data set that may include one or more of the first data set or the second data set. Training of the machine learning model may be performed by performing an epoch of training of the machine learning model. Each epoch may correspond to a forward pass and a backward pass associated with training the machine learning model using each data point of the combined training data set. Training of the machine learning model may be performed using a number of additional epochs of training. The augmentation techniques and the relevant details to generate the second, third, and fourth data set may remain the same as those described with regards to blocks 206 through 216 of FIG. 2.

FIG. 3 depicts a flow diagram of one example of a method 300 for predicting a likelihood of a health condition to occur, in accordance with one or more aspects of the present disclosure. The method is performed by processing logic that may comprise hardware (circuitry, dedicated logic, etc.), software (such as is run on a general purpose computer system or a dedicated machine), or a combination thereof In one implementation, the method is performed using the computing device 160 and trained machine learning model 150 of FIG. 1, while in some other implementations, one or more blocks of FIG. 3 may be performed by one or more other machines not depicted in the figures.

For simplicity of explanation, methods are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events. Additionally, it should be appreciated that the methods disclosed in this specification are capable of being stored on an article of manufacture to facilitate transporting and transferring such methods to computing devices. The term article of manufacture, as used herein, is intended to encompass a computer program accessible from any computer-readable device or storage media.

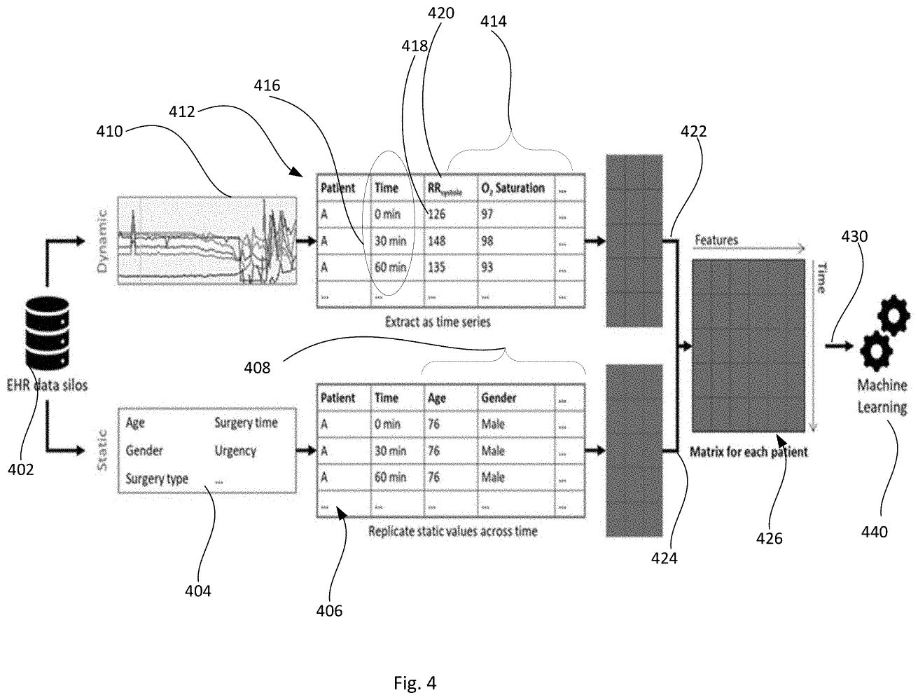

At block 302, a first set of data comprising static data may be received for one or more first set of features of health data associated with a patient. An example of a first set of data comprising static data is depicted in FIG. 4. The static data may be obtained from a data store (e.g., database) 402. Data store 402 may be comparable to one or more repositories 110A-N. Static data 404 may represent data not likely to change over the time interval of interest. The time interval of interest may be, for example, during hospitalization of a patient. The static data 404 may be collected in a categorical format suitable for some EHR systems but may not be in a format appropriate for use with a machine learning model. The static data 404 may be transformed into an appropriate format for neural networks, such as, in a tabular or column format as shown in table 406. The first set of data comprising the static data may include a first set of features 408 for the patient. For example, the features related to the patient's physical attributes may include age, gender, height, weight, etc. Additionally, some one time treatment related data may be included in the first set of data. The treatment features may include, but not be limited to, anesthesia type, anesthesia score, cardioplegic solution, aortic cross clamp time, anesthetic monitoring time, surgery duration, surgery type, urgency, etc. as related to cardiothoracic surgery domain. In another medical domain, the features may be different. For example, in neurosurgical domain, other features than the listed features may be more appropriate, although there may be some overlaps between domains as well.

At block 304, a second set of data may be received for the patient. The second set of data may comprise dynamic data for one or more second set of features of health data associated with the patient. Each value corresponding to each feature of the second set of features may correspond to one of a plurality of time values (e.g., time point, time interval, etc.). In some example, the second set of data may be collected using one or more medical devices. In other examples, the second set of data may be collected by an individual (e.g., a health care professional) at each of the plurality of time values. In some examples, a combination of medical devices and individuals may collect the second set of data over different time intervals.

An example of a second set of data comprising dynamic data is depicted in FIG. 4. The dynamic data may be obtained from the data store 402, which may be comparable to one or more repositories 110A-N. Dynamic data 410 may represent data likely to change over time, as depicted by the plot showing peaks and valleys in the values of the dynamic data 410. The dynamic data 410 may be transformed into an appropriate format for neural networks, such as, in a tabular or column format as shown in table 412. The second set of data comprising the dynamic data may include a second set of features 414 for the patient. Values for each feature may correspond to a plurality of time values 416. For example, the plurality of time values is shown to be in intervals of 30 minutes for the dynamic data 410 in table 412. The plurality of time values 416 include a time value at 0 minute, 30 minute, 60 minute, and so on. Each value (for example, value 418) corresponding to each feature (for example, feature 420) of the second set of features may correspond to one of the plurality of time values 416 (for example, time value of “0 minute”). Thus, in the example, the data set indicates that at time 0 minute, the value of feature “RR Systole” is “126.” At time 30 minute, the value of feature “RR Systole” is tabulated as “148.”

By way of example only, the second set of features 414 may include, but not be limited to, vital signs, such as Systolic, mean and diastolic arterial pressure, Systolic, mean and diastolic pulmonary artery pressure, central venous pressure, ventilator FiO₂ setting, heart and respiratory frequency, body temperature. In additional example, the second set of features 414 may include, but not be limited to, arterial blood gas (BGA) features, such as Bicarbonate, Glucose, Haemoglobin, Oxygen Saturation, Partial Pressure of Carbon Dioxide and Oxygen, PH Level, Potassium, Sodium. In further example, the second set of features 414 may include, but not be limited to, laboratory results, such as, Albumin, Bilirubin, Urea, C-Reactive Protein, Creatine Kinase, Gamma-Glutamyltransferase, Glutamic Oxaloacetic Transaminase, Hemoglobin , Hematocrit, International Normalized Ratio, Kreatinin, Leukocytes, Lactate Dehydrogenase, Magnesium, Partial Thromboplastin Time, Platelets, Prothrombin Time, and balance output, such as, Bleeding Rate, Urine Flow Rate, etc.

In some implementations, imaging data may be used for the features of dynamic data. For example, textual data from reports of chest x-rays and echocardiograms may be extracted to obtain key findings, and may be incorporates as features. In some examples, the imaging reports are scans of text documents, in which case an OCR (optical character recognition) may be run on these documents first. Some of the features may include, for chest x-rays for example, Radio-transparency of the lung (left and right), Fluid collection in the pleural cavity (L+R), Presence of chest wall hematoma, Mediastinal widening, Free air in the abdominal cavity, Pneumothorax, etc. For echocardiograms, the features may include Ventricular function (L+R), Pericardial Effusion, Major valve regurgitation (aortic, tricuspid, and mitral valve), Atrial and Ventricular filling (L+R), etc.

At block 306, a time series data set may be derived based on the first set of data and the second set of data. The first set of data may be replicated for each of the plurality of time values. As shown in FIG. 4, the first set of data for patient “A” included age value of “76” and gender value of “male” and these values are likely to be unchanged for the time interval of interest. The first set of data 404 is then replicated for each of the plurality of time values 416. Thus, table 408 may also include the plurality of time values 416 and corresponding values for features 408, where each value of the features 408 has been replicated for each time value of the plurality of time values 416. As can be seen, the values for age as “76” and gender as “male” has been replicated for each time value 0 min, 30 min, 60 min, etc. The first set of data 404 and the second set of data 410 corresponding to each time value (e.g., 0 min, 30 min, 60 min, and so on) of the plurality of time values 416 is then combined, as shown with arrows 422 and 424, to derive a feature matrix. The feature matrix thus includes a time series data set 426 based on all features of the first set of data 404 and second set of data 410 for each time value of the plurality of time values 416. The time series data may be derived as time progresses.

At block 308, a value corresponding to a feature may be determined as being absent in the time series data set. For example, a set of laboratory results may have been measured for a time value at 0 minute, however, the results may not have been measured for the next several time intervals, including at 30 minutes, 60 minutes, etc. As such, there may not have been any results corresponding to these time values in data store 402 and the values in the time series may be missing. The system may determine that the values corresponding to the feature are absent for the time series data at the time values 30 minutes and 60 minutes (and possibly for more time intervals). In another example, a health care professional may have measured the blood pressure of a patient at 0 minute, 30 minute, and 60 minute, 120 minute, but did not measure it at 90 minute. Thus, the time series data may be missing the blood pressure value at 90 minute. In another example, there may be a technical glitch leading to a value being omitted from the time series data even though a value corresponding to the feature may have been collected. Thus, one or more values corresponding to one or more features may be determined as absent in the time series data set 426.

At block 310, the value for the feature may be assigned (e.g., imputed) using a given data. In some example, the given value may comprise a previously measured value of the feature. In some examples, the given value may comprise a clinically acceptable value for the feature. In the example where the laboratory results were not collected for 30 minutes and 60 minutes, the previously measured or available data may be used as the given value to assign to the value for the results. Thus, the results available for 0 minute may be used for 30 minute and 60 minute time values. In another example, the values for a feature may have been expected to and have in fact been steadily increasing for a series of time values. In such a case, a given value may be selected based on the clinically accepted value at the missing time point. An indicator (e.g., a label) corresponding to the value may be added indicating that the value for the feature is an assigned (e.g., imputed) value. The indicator may be helpful for the machine learning algorithm as it indicates the data was originally missing and the machine learning algorithm can take the occurrence of missing data into consideration for learning the pattern appropriately.

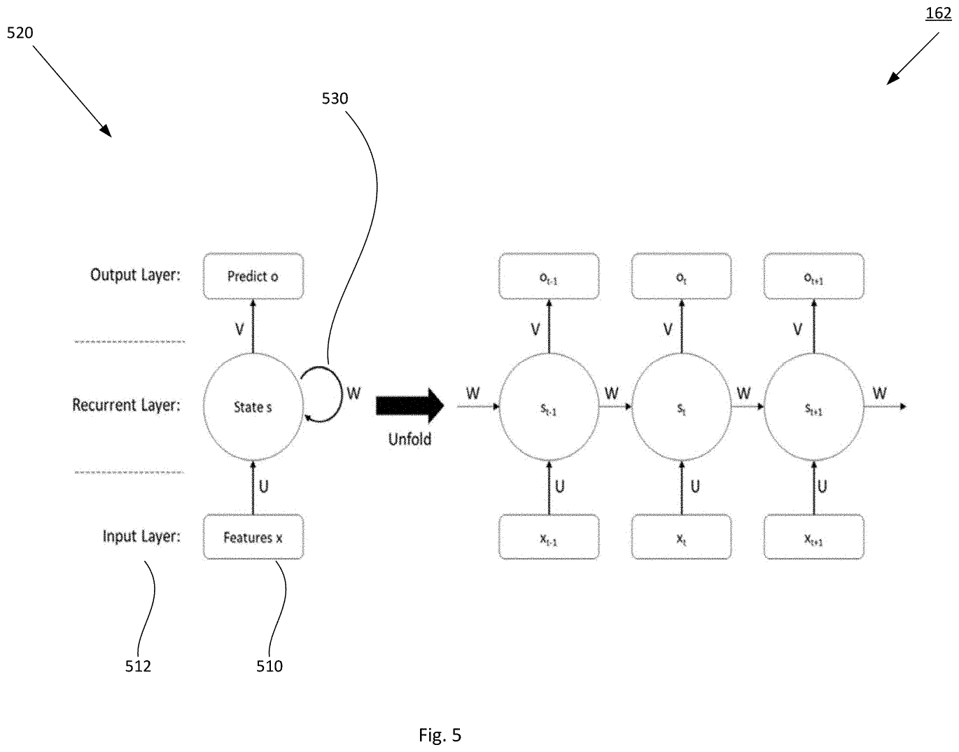

At block 312, the time series data set may be provided as input to a trained machine learning model to predict health conditions of the patient. Furthermore, likelihood may be predicted of one or more conditions to occur corresponding to one or more future time values for the patient. FIG. 4 shows that the time series data 426 is provided via arrow 430 to machine learning model 440 (e.g., comparable to model 150 of FIG. 1). FIG. 5 further shows an example of prediction of health conditions using a machine learning model. In the example, a gated recurrent unit (GRU) network is used. Prediction engine 162 may compute an internal state Sₜ at each point in time t, based on input features Xₜ. The features Xₜ may be passed to the prediction engine 162 as time progresses. At each point in time, a likelihood Oₜ of a specific condition (e.g., renal failure) to occur may be computed. As shown in FIG. 5, features×510 may be provided as input to the input layer 512 of model 520. Model 520 may be same or similar to model 150 of FIG. 1. The time series data set 426 of FIG. 4 may be provided as Features×510 to the model 520. Variables U, V, and W may represent numeric matrices for weights of parameters for use in the different layers of the machine learning model. The machine learning model may learn these matrices, for example by means of backpropagation, etc. In the example of the unfolded model 540, the matrix U takes as input Features Xₜ at time “t” and projects the features onto the internal state Sₜ. Internal state Sₜ is also informed by the previous state Sₜ₋₁ at time “t-1” which is projected to Sₜ through the matrix W. The internal state Sₜ includes the total knowledge about the patient at this time “t.” An outcome Oₜ for this time “t” may be projected through matrix V. Matrix V may project the internal state Sₜ in the form of an output (e.g., chance of complication, class of tumor, etc.).

At block 314, a likelihood of a condition to occur at a future time value may be determined to be above a predefined threshold. For example, a threshold may be defined in the system for each of the conditions that the machine learning system is designed to predict. If the likelihood of a certain condition to occur is more than the predefined threshold, it may indicate that a decision needs to be made regarding the treatment of the patient. It may also indicate the success of failure or an ongoing therapy or intervention. Thus, it may serve as a therapy monitoring device. In addition, the likelihood values may be continuously projected on a screen to health care professionals. For example, at a physician or nurse's station, a screen may project the likelihood of conditions of a number of patients. At a screen near a particular patient, only the likelihood values of the particular patient may be displayed. Even if the likelihood values do not surpass the predefined threshold, a health care professional may decide to modify the treatment plans and/or diagnostic plan based on monitoring the likelihood values on the screen.

In some embodiments, health care professionals may decide, based on the likelihood being above the predefined threshold, that a corrective action is to be performed. In that case, a health care professional may perform a corrective action. The health care professional may perform one or more of a plurality of corrective actions. In one embodiment, the corrective action may comprise administering an active agent to treat the condition. In another embodiment, the corrective action may comprise performing an operative procedure on the patient. In another embodiment, the corrective action may comprise avoidance of selected active agents. In another embodiment, the corrective action may comprise modifying (e.g., increasing, decreasing, etc.) of a dose of an existing active agent therapy. In another embodiment, the corrective action may comprise initiation of a diagnostic test. The diagnostic test may be used to further diagnose the predicted conditions or additional conditions. In another embodiment, the corrective action may comprise modifying the level of patient monitoring. For example, patients may be monitored using a non-invasive technique (e.g., non-invasive pressure monitoring, non-invasive pulseoxymetry sensor, etc.) when a low level monitoring scheme is used. At a different level, invasive procedures (e.g., invasive arterial line) may be used to monitor the patient. At another level, the invasive procedure may be of a higher intensity level, such as a pressure sensing catheter in the pulmonary artery, etc. Other types of additional medical interventions may be performed to treat the condition predicted using the machine learning model. Clinical judgment by the health care professional team may be an important aspect of deciding the corrective action to the undertaken.

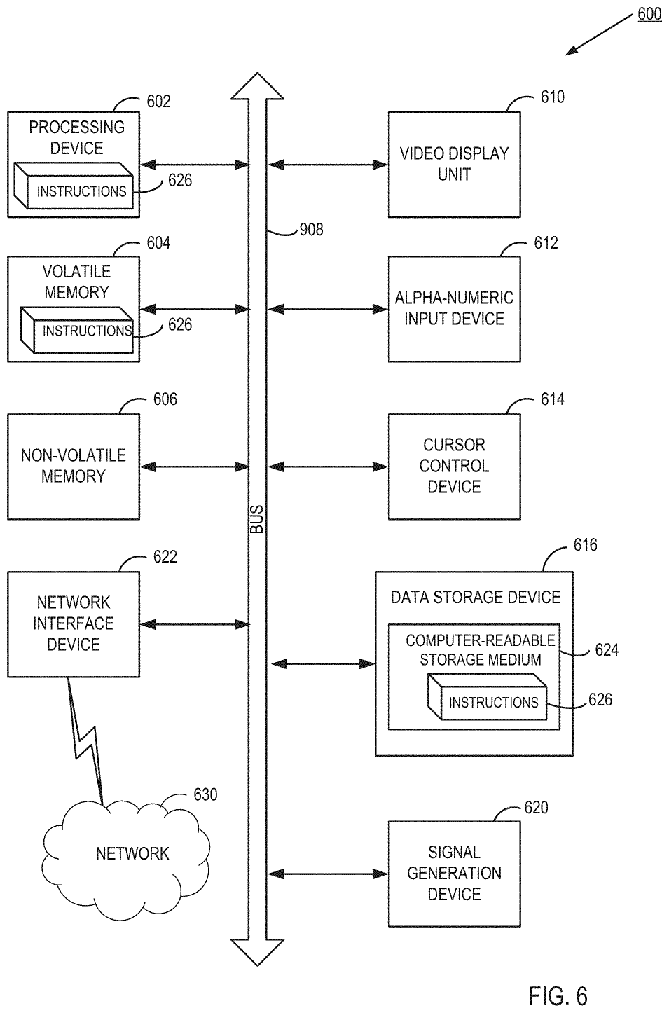

FIG. 6 depicts a block diagram of an illustrative computer system 600 operating in accordance with one or more aspects of the disclosure. In various illustrative examples, computer system 600 may correspond to a computing device within system architecture 100 of FIG. 1. In certain implementations, computer system 600 may be connected (e.g., via a network 630, such as a Local Area Network (LAN), an intranet, an extranet, or the Internet) to other computer systems. Computer system 600 may operate in the capacity of a server or a client computer in a client-server environment, or as a peer computer in a peer-to-peer or distributed network environment. Computer system 600 may be provided by a personal computer (PC), a tablet PC, a set-top box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein.

In a further aspect, the computer system 600 may include a processing device 602, a volatile memory 604 (e.g., random access memory (RAM)), a non-volatile memory 606 (e.g., read-only memory (ROM) or electrically-erasable programmable ROM (EEPROM)), and a data storage device 616, which may communicate with each other via a bus 608.

Processing device 602 may be provided by one or more processors such as a general purpose processor (such as, for example, a complex instruction set computing (CISC) microprocessor, a reduced instruction set computing (RISC) microprocessor, a very long instruction word (VLIW) microprocessor, a microprocessor implementing other types of instruction sets, or a microprocessor implementing a combination of types of instruction sets) or a specialized processor (such as, for example, an application specific integrated circuit (ASIC), a field programmable gate array (FPGA), a digital signal processor (DSP), or a network processor).

Computer system 600 may further include a network interface device 622. Computer system 600 also may include a video display unit 610 (e.g., an LCD, a touch enabled display unit, etc.), an alphanumeric input device 612 (e.g., a keyboard), a cursor control device 614 (e.g., a mouse), and a signal generation device 620.

Data storage device 616 may include a non-transitory computer-readable storage medium 624 on which may store instructions 626 encoding any one or more of the methods or functions described herein, including instructions for implementing methods 200 and 300 of FIGS. 2 and 3, respectively.

Instructions 626 may also reside, completely or partially, within volatile memory 604 and/or within processing device 602 during execution thereof by computer system 600, hence, volatile memory 604 and processing device 602 may also constitute machine-readable storage media.

While computer-readable storage medium 624 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

The methods, components, and features described herein may be implemented by discrete hardware components or may be integrated in the functionality of other hardware components such as ASICS, FPGAs, DSPs or similar devices. In addition, the methods, components, and features may be implemented by component modules or functional circuitry within hardware devices. Further, the methods, components, and features may be implemented in any combination of hardware devices and computer program components, or in computer programs.

Unless specifically stated otherwise, terms such as “generating,” “providing,” “training,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. Also, the terms “first,” “second,” “third,” “fourth,” etc. as used herein are meant as labels to distinguish among different elements and may not have an ordinal meaning according to their numerical designation.

Examples described herein also relate to an apparatus for performing the methods described herein. This apparatus may be specially constructed for performing the methods described herein, or it may comprise a general purpose computer system selectively programmed by a computer program stored in the computer system. Such a computer program may be stored in a computer-readable tangible storage medium.

The methods and illustrative examples described herein are not inherently related to any particular computer or other apparatus. Various general purpose systems may be used in accordance with the teachings described herein, or it may prove convenient to construct more specialized apparatus to perform methods 200 and 300 and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

1. A system comprising:
a memory; and
a processor, coupled to the memory, the processor to:
obtain a first data set comprising one or more features of health data associated with one or more patients to train a machine learning model to predict health conditions of patients, the one or more features corresponding to a plurality of time values;
generate a second data set by one or more of:
i) applying a bias value to values of a first feature of the first data set;
ii) removing one or more data points from the first data set; or
iii) modifying a length of a time interval comprising the plurality of time values; and
perform training of the machine learning model using a training data set comprising one or more of the first data set or the second data set.

2. The system of claim 1, wherein to perform training of the machine learning model, the processor is to perform an epoch of training the machine learning model;

3. The system of claim 2, wherein the epoch corresponds to a forward pass and a backward pass associated with training the machine learning model using each data point of the training data set.

4. The system of claim 1, wherein to perform training of the machine learning model, the processor is further to perform a number of additional epochs of training.

5. The system of claim 1, wherein the processor is further to:
identify an outcome associated with the one or more features of health data; and
add a label identifying the outcome associated with the one or more features to the first data set.

6. The system of claim 1, wherein a bias value corresponds to a difference applied to the values of the first feature of the first data set.

7. The system of claim 1, wherein to apply the bias value, the processor is to:
append a random value to the values of the first feature of the first data set.

8. The system of claim 7, wherein the random value is within a specified range of values.

9. The system of claim 7, wherein the random value is within a specified number of standard deviation of the values of the first feature.

10. The system of claim 1, wherein applying the bias value results in each of the values of the first feature to remain within a clinically acceptable range of values for the first feature.

11. The system of claim 1, wherein to apply the bias value, the processor is to:
formulate a set of clinically plausible values for the first feature; and
replace the values of the first feature of the first data set using one or more random values from the set of clinically plausible values.

12. The system of claim 1, wherein to remove the one or more data points from the first data set, the processor is to:
remove a particular value of a second feature of the first data set.

13. The system of claim 1, wherein to remove the one or more data points from the first data set, the processor is to:
remove each value of a third feature of the first data set.

14. The system of claim 1, wherein to remove the one or more data points from the first data set, the processor is to:
remove values of each of the one or more features of the first data set corresponding to a specified time interval.

15. The system of claim 1, wherein to remove the one or more data points from the first data set, the processor is to:
randomly remove one or more values of a fourth feature of the first data set corresponding to each of the one or more patients.

16. The system of claim 1, wherein to modify the length of the time interval, the processor is to:
increase the length of the time interval.

17. The system of claim 1, wherein to modify the length of the time interval, the processor is to:
decrease the length of the time interval.

18. The system of claim 1, wherein to modify the length of the time interval, the processor is to:
for each particular feature of the one or more features:
generate a mathematical function identifying a relationship between the plurality of time values and values of the particular feature using linear interpolation; and
generate values for a second plurality of time values using the mathematical function.

19. A system comprising:
a memory; and
a processor, coupled to the memory, the processor to:
obtain a first data set comprising one or more features of health data associated with one or more patients to train a machine learning model to predict health conditions of patients;
perform a first epoch of training using the first data set to train the machine learning model;
upon completing the performance of the first epoch, generate a second data set by applying a bias value to values of a first feature of the first data set; and
perform a second epoch of training using the second data set to train the machine learning model.

20-36. (canceled)

37. A method comprising:
receiving a first set of data comprising static data for one or more first set of features of health data associated with a patient;
receiving a second set of data comprising dynamic data for one or more second set of features of health data associated with the patient, wherein each value corresponding to each feature of the second set of features corresponds to one of a plurality of time values;
deriving a time series data set based on the first set of data and the second set of data;
determining that a value corresponding to a feature is absent in the time series data set;
assigning the value for the feature using a given data;
adding an indicator corresponding to the value indicating that the value for the feature is an assigned value; and
providing the time series data set as input to a trained machine learning model to predict health conditions of the patient.

38-53. (canceled)
